(19) United States
(12) Patent Application Publication
(10) Pub. No.: US 2014/0303636 A1
(43) Pub. Date: Oct. 9, 2014

(54) Intraocular Lens Injector Cartridge Providing Lens Control

(71) Applicant: Bausch & Lomb Incorporated, Rochester, NY (US)

(72) Inventor: Moises A. Valle, Tustin, CA (US)

(21) Appl. No.: 13/859,088

(22) Filed: Apr. 9, 2013

(51) Int. Cl. A61F 9/007

(52) U.S. Cl. CPC A61F 9/007
USPC 606/107

(57) ABSTRACT

An intraocular lens (IOL) cartridge, comprising a lumen wall having a distal end and a proximal end, and defining a rigid lumen portion having a longitudinal axis extending therethrough, the lumen wall having a first ridge segment disposed on an inner surface thereof and extending in the direction of the longitudinal axis, a first wing coupled to the lumen wall at the proximal end and having a first ridge portion extending therefrom, and a second wing coupled to the lumen wall at the proximal end and having a second ridge portion extending therefrom, the first wing is coupled to the second wing by a hinge. When the first wing and the second wing are moved to a closed position to form a second lumen portion, the first ridge portion and the second ridge portion form a second ridge segment, the first ridge segment and the second ridge segment align in the direction of the longitudinal axis.

### FIELD OF INVENTION

The present invention relates to intraocular lens injector cartridges, and more particularly to intraocular lens injector cartridges providing lens control.

### BACKGROUND OF THE INVENTION

Intraocular lenses (also referred to herein as IOLs or simply as lenses) are artificial lenses used to replace or supplement natural crystalline lenses of eyes when the natural lenses are diseased or otherwise impaired. IOLs may be placed in either the posterior chamber or the anterior chamber of an eye.

IOLs come in a variety of configurations and materials. Various instruments and methods for implanting such IOLs in an eye are known. Typically, an incision is made in a patient's cornea and an IOL is inserted into the eye through the incision. In one technique, a surgeon uses surgical forceps having opposing blades to grasp the IOL and insert it through the incision into the eye. While this technique is still practiced today, more and more surgeons are using IOL injectors which offer advantages such as affording a surgeon more control when inserting an IOL into an eye and permitting insertion of IOLs through smaller incisions. Smaller incision sizes (e.g., less than about 3 mm) are preferred over larger incisions (e.g., about 3.2 to 5+ mm) since smaller incisions have been associated with to reduced post-surgical healing time and reduced complications such as induced astigmatism.

Injectors come in many configurations, for example an injector may be configured such that the IOL is loaded directly into the body of the injector. Alternatively, an injector may comprises a cartridge into which an IOL is loaded and an injector body into which the cartridge, with the IOL inside, is loaded.

Conventional IOL cartridges include a load chamber connected to a nozzle. In some configurations, the nozzle includes a small diameter distal end that is suitable for insertion into the eye for delivery of the IOL into the eye. After mating the cartridge with the injector body, a plunger may be slid (e.g., translated) or screwed through the lumen of the cartridge to urge the IOL through the load chamber and the nozzle into an eye.

In order for an IOL to fit through an incision, it is typically folded and/or compressed prior to entering an eye where it will assume its original unfolded/uncompressed shape. Folding and compression can occur prior to, during and/or after the IOL is loaded into the cartridge (e.g., using forceps or as a result of movement through a tapered nozzle). Since IOLs are very small and delicate articles of manufacture, great care must be taken in their handling, both as they are loaded into an injector and as they are injected into patients' eyes.

It is important that an IOL be expelled from the distal end of the IOL injector and into the eye in an undamaged condition and in a predictable orientation. Should an IOL be damaged or expelled from the injector in an incorrect orientation, a surgeon may need to remove or further manipulate the IOL in the eye, possibly resulting in trauma to the surrounding tissues of the eye. To achieve proper delivery of an IOL, consistent loading of the IOL into the injector device with a minimum opportunity for damaging the IOL is desirable.

In general, the IOL is provided to the surgeon in packaging, such as a vial, plastic blister package, or other container for maintaining the IOL in a sterile condition. The IOL is removed from the packaging and loaded into the load chamber of the cartridge prior to insertion into the patient's eye. Removal of the IOL from the packaging and transfer to the load chamber is usually accomplished with forceps or a similar device. The forceps may simply be used to place the IOL on or in the load chamber of the cartridge, or may also fold the IOL to a reduced size for injection into the eye.

Certain problems may be encountered during delivery of the IOL from the cartridge and into an eye. For instance, during movement though the cartridge lumen, the orientation of the optic and haptic portions may be difficult to control especially if the IOL to fold or compress the IOL as it is moved through the nozzle. In addition, problems may be encountered during engagement of the tip of the plunger with the IOL, resulting in damage of optic, haptics, or both.

In view of the above, there is a need for a cartridge that more effectively receives and manages passage of an IOL therethrough.

### SUMMARY

Aspects of the present invention are directed to an intraocular lens (IOL) cartridge, comprising a lumen wall having a distal end and a proximal end, and defining a rigid lumen portion having a longitudinal axis extending therethrough, the lumen wall having a first ridge segment disposed on an inner surface thereof and extending in the direction of the longitudinal axis, a first wing coupled to the lumen wall at the proximal end and having a first ridge portion extending therefrom, and a second wing coupled to the lumen wall at the proximal end and having a second ridge portion extending therefrom, the first wing is coupled to the second wing by a hinge. When the first wing and the second wing are moved to a closed position to form a second lumen portion, the first ridge portion and the second ridge portion form a second ridge segment, the first ridge segment and the second ridge segment align in the direction of the longitudinal axis.

In some embodiments, the first wing is rigidly coupled to the lumen wall. The first wing may be directly connected to the lumen wall.

In some embodiments, the second ridge portion is disposed opposite the hinge when the first wing and the second wing are in a closed position.

The distal end of the first ridge segment may be terminated proximally of the distal end of the rigid lumen portion.

The proximal end of the first ridge segment and the distal end of the second ridge segment may be in contact with one another when the first wing and the second wing are in the closed position.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a projection view of an embodiment of an intraocular lens injector cartridge according to aspects of the present invention from a location proximal to the cartridge and with the cartridge in an open configuration;

FIG. 2 is a projection view of the cartridge of FIG. 1 from a location proximal to the cartridge and with the cartridge in a closed configuration;

FIG. 3 is a cross-sectional side view of the injector of FIG. 1 taken along line 3-3 in FIG. 2;

FIG. 4 is a projection view of an embodiment of an intraocular lens injector cartridge according to aspects of the present invention from a location proximal to the cartridge, with the cartridge in an open configuration, and with an IOL disposed therein;

FIG. 4A is a section view taken along line 4A-4A in FIG. 4 showing the lumen wall defining the rigid lumen portion;

FIG. 5 is projection view of the cartridge of FIG. 1 from a location proximal to the cartridge, with the cartridge in a closed configuration, and with an IOL disposed therein;

FIG. 6 is a schematic illustration of an injector kit including the cartridge of FIG. 1 and an injector body in a disassembled state; and

FIG. 7 is a schematic illustration of a combination of the cartridge of FIG. 1 and an injector body (i.e., with the kit of FIG. 4 in an assembled state);

### DETAILED DESCRIPTION

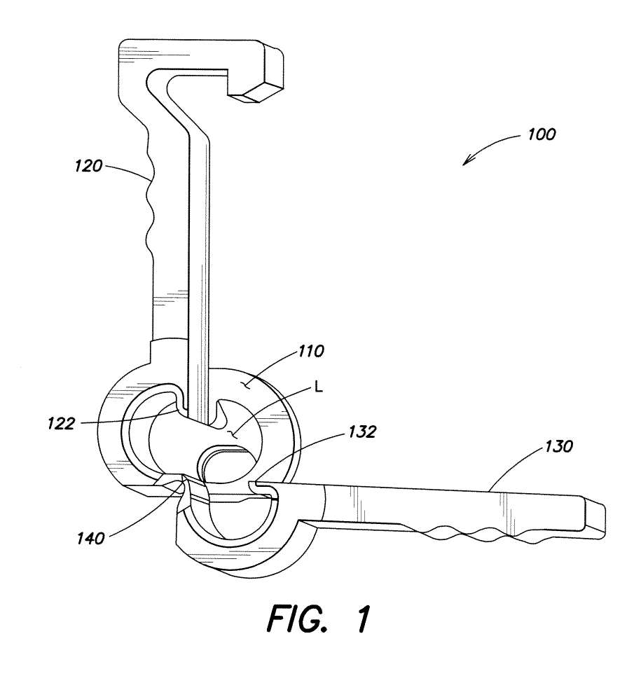

Aspects of the invention will be discussed below with reference to FIGS. 1-3. An intraocular lens (IOL) cartridge 100 comprises a lumen wall 110 defining a rigid lumen portion L, and a first wing 120 and a second wing 130.

Lumen wall 110 has a distal end 112 and a proximal end 114. The term “distal” means tending toward the end of the injector body where the lens is delivered into an eye. The term “proximal” means tending toward the end of the injector body that is opposite from where the lens is delivered from the injector into an eye.

Rigid lumen portion L has a longitudinal axis LA extending therethrough. The rigid lumen portion has fixed dimensions as determined by the dimension of the rigid structure from which it is made. Lumen wall 110 has a first ridge segment 116 disposed on an inner surface thereof that extends in the direction of the longitudinal axis.

First wing 120 is coupled to lumen wall 110 at the proximal end 114. First wing has a first ridge portion 122 extending therefrom.

Second wing 130 is coupled to the lumen wall 110 at proximal end 114. Second wing 130 has a second ridge portion 132 extending therefrom. The first wing is hingedly coupled to the second wing at hinge 140.

Typically, the first wing is rigidly coupled and directly connected to lumen wall 110. However, for example, the first wing may be rigidly or flexibly coupled to the lumen wall through the hinge. For example, the second wing may be directly coupled to lumen wall 110 (e.g., through the hinge) or the second wing may be coupled to the lumen wall though through the hinge and first wing.

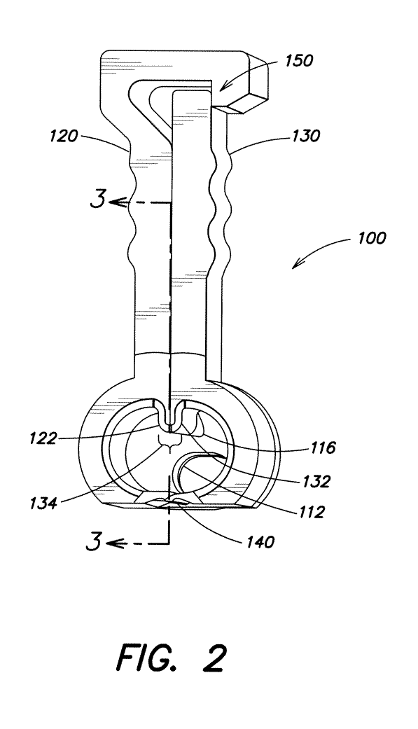

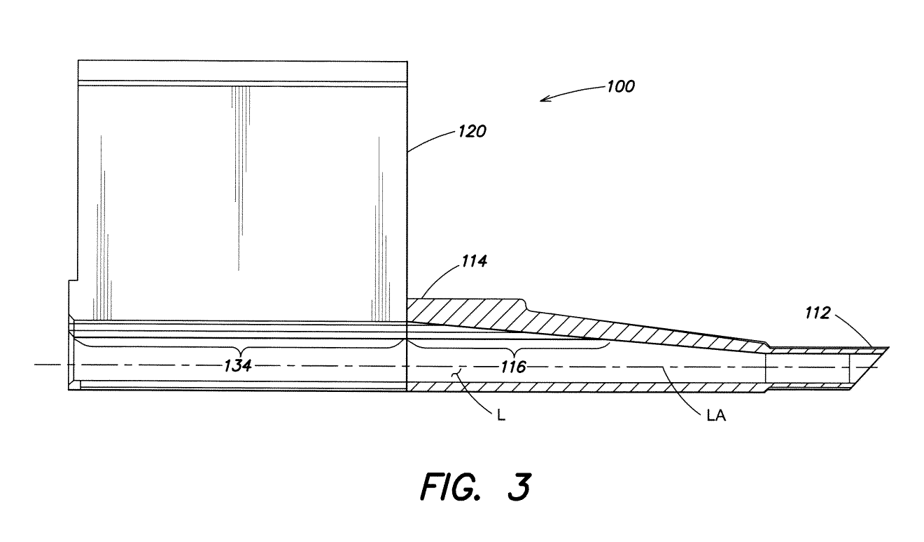

As shown in FIG. 2, when the first wing and the second wing are moved to a closed position to form a second lumen portion, first ridge portion 122 and second ridge portion 132 form a second ridge segment 134. As shown in FIGS. 2 and 3, the first ridge segment and the second ridge segment align in the direction of the longitudinal axis. The second ridge segment is disposed opposite hinge 140 when the first wing and the second wing are in a closed position. In some embodiments, the second lumen portion has an oval cross sectional shape which tends to provide additional edge control during the folding an injection processes; however other shapes such a circular may also be used with the ridge structure. Additionally, the cross section of the second lumen portion may transition in shape, for example from an oval shape at the proximal end to a circular shape or more circular shape at the distal end.

In some embodiments, the cartridge locks in a closed position. As shown in FIG. 2, the outer portions of the first wing and the second wing form a lock 150. The first wing and second wing snap to a closed position and interference maintains the cartridge in the closed position; however, any suitable technique for maintaining the cartridge in a closed position may be used.

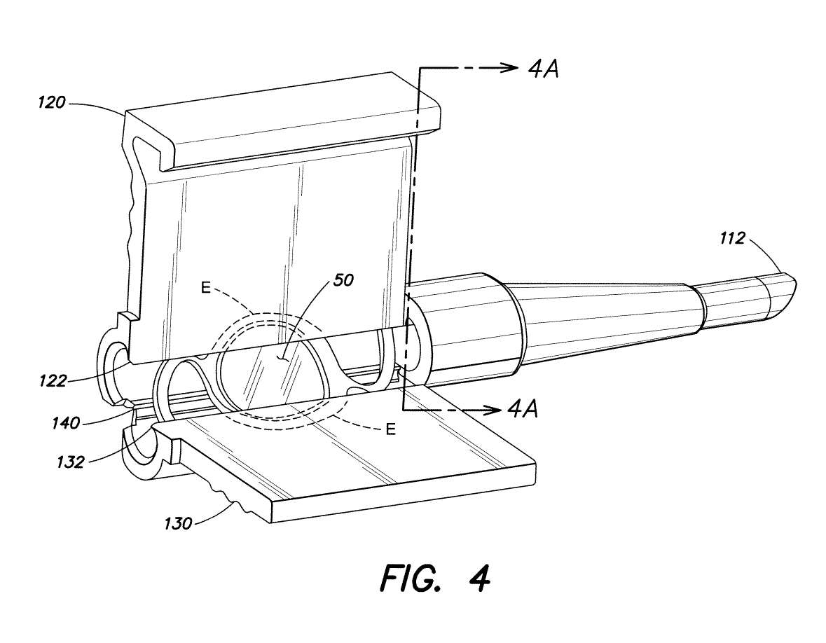

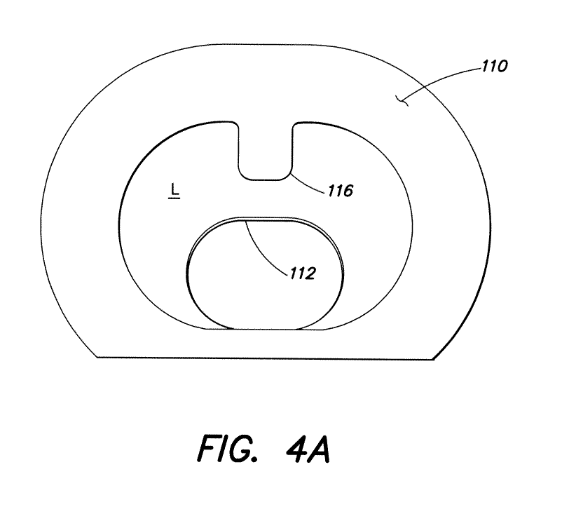

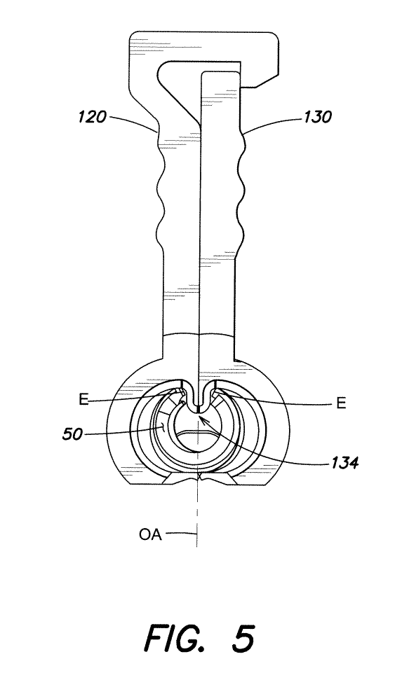

As shown in FIG. 4, a lens 50 is loaded onto the first wing 120 and second wing 130 with the edge E of the IOL positioned under first ridge portion 122 and the second ridge portion 132. The first ridge portion and the second ridge portion operate in the manner of conventional ridge portions to control edge E of the lens during the loading and folding process. As shown in FIG. 5, upon closure of the cartridge, lens 50 attains a folded configuration with edge E disposed in the anticipated positions proximate opposite sides of the second ridge segment 134. The edge E may contact the opposite sides as it travels through lumen L or be in close proximity to reduce rotation or likelihood of rotation of the lens as the plunger is actuated.

FIG. 4A is a section view taken along line 4A-4A in FIG. 4 showing the lumen wall 110 defining the rigid lumen portion L and distal end 112 though which an IOL is delivered into an eye.

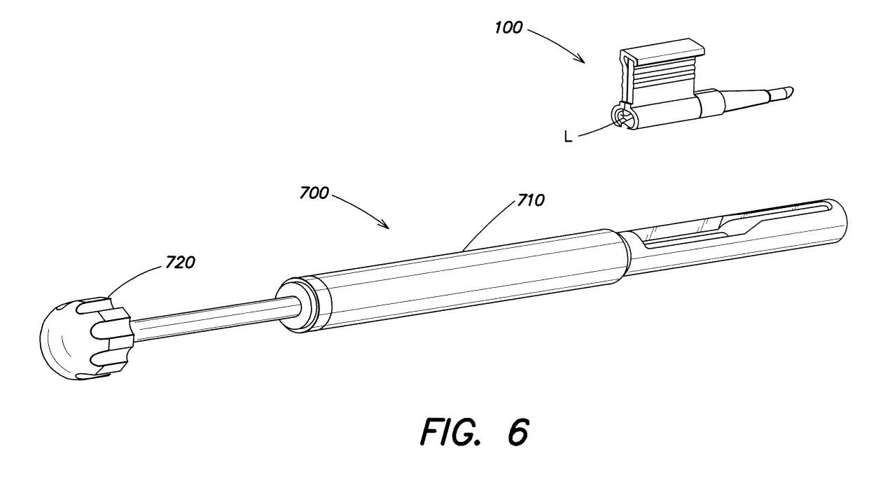

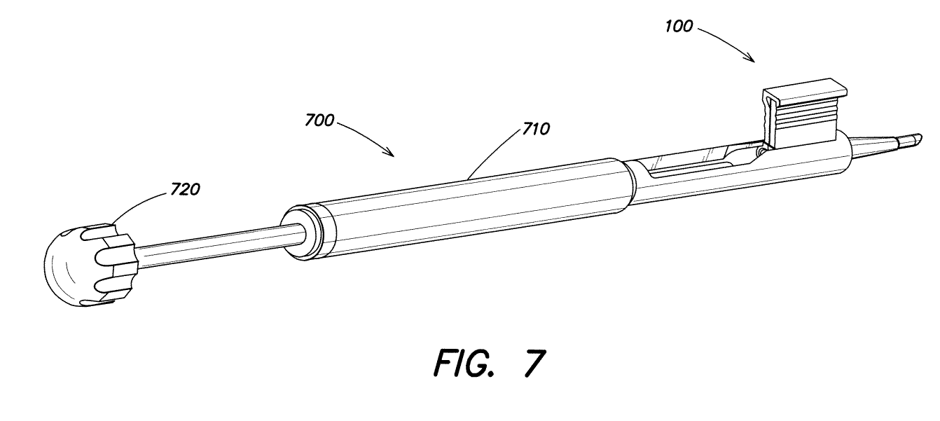

FIGS. 6 and 7 are schematic illustrations of an injector kit 700 including cartridge 100 as described above with reference to FIG. 1 and an injector body 710, with the cartridge and the injector body in a disassembled state and the cartridge and the injector body in an assembled state, respectively. Injector kit 700 includes a plunger 720 for moving an IOL through lumen L of cartridge 100. The plunger can be configured to be actuatable in a screw-type or syringe-type manner.

Upon actuation of plunger 720, the lens is moved distally through the lumen with edge E of the lens remaining in contact or close proximity with the opposite sides of the second ridge segment and the first ridge segment as it progresses. As a result, lens rotation is reduced or prevented as the lens progresses through the lumen during actuation of the plunger. Some minor amount of lens rotation may occur during actuation of the plunger with deleterious effects. For example, a sufficient result can be achieved if rotation about the optical axis OA of the lens is less than about 10-15 degrees.

Referring again to FIGS. 1-3, the inventor has determined that, by providing first ridge segment 116 in the rigid lumen portion, the first ridge segment complements the anti-lens-rotation effect of the second ridge segment 134, and lens rotation can be appropriately prevented during the entirety of the injection process from loading of the IOL, movement through the winged portion of the cartridge, until sufficient compression of the IOL occurs, thereby controlling orientation of the optic and haptic portions of the IOL and reliably delivering the lens into an eye through open distal end 112.

It will be appreciated that the height of the ridge portions are selected to be tall enough to avoid slipping of the lens edge off the ridge portions 122, 132 and short enough to avoid any interference of the ridge portions with plunger 720 during the IOL injection process. It will also be appreciated that the ridge need only have a sufficient height until the lens is folded to a point at which lens is no longer susceptible to rotation. An adequate amount of folding for a lens to avoid undue rotation, for a given lens design to be used in a given lumen design can be determined empirically. As shown in FIG. 3, the height of first ridge segment 116 may be tapered along the direction of the longitudinal axis LA at its distal end such that the ridge height is reduced and, in some embodiments, terminated at a location proximal to the open, distal end 112 of lumen L.

It is generally advantageous if, when the cartridge is in a closed position, the proximal end of the first ridge segment and the distal end of the second ridge segment are in contact with one another and if the first ridge segment and second ridge segment, together, form a continuous length so that the IOL moves smoothly through the lumen during the injection process. Such a configuration avoids a haptic or edge portion E of the IOL from being impeded during movement through lumen L thereby avoiding damage to the IOL. It will be appreciated that a small gap relative to the dimensions of the IOL may be present without giving rise to a possibility of impediment and damage to the IOL.

In some embodiments, the first ridge segment and the second ridge segment form a continuous outer ridge surface. However, in some embodiments, the outer dimensions of the proximal end of first ridge segment are different than outer dimensions of the distal end of the second ridge segment.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An intraocular lens (IOL) cartridge, comprising:
a lumen wall having a distal end and a proximal end, and defining a rigid lumen portion having a longitudinal axis extending therethrough, the lumen wall having a first ridge segment disposed on an inner surface thereof and extending in the direction of the longitudinal axis;
a first wing coupled to the lumen wall at the proximal end and having a first ridge portion extending therefrom; and
a second wing coupled to the lumen wall at the proximal end and having a second ridge portion extending therefrom, the first wing is coupled to the second wing by a hinge,
wherein, when the first wing and the second wing are moved to a closed position to form a second lumen portion, the first ridge portion and the second ridge portion form a second ridge segment, the first ridge segment and the second ridge segment align in the direction of the longitudinal axis.

2. The cartridge of claim 1, wherein the first wing is rigidly coupled to the lumen wall.

3. The cartridge of claim 2, wherein the first wing is directly connected to the lumen wall.

4. The cartridge of claim 1, wherein the second lumen portion has as an oval cross sectional shape.

5. The cartridge of claim 1, wherein the second ridge portion is disposed opposite the hinge when the first wing and the second wing are in the closed position.

6. The cartridge of claim 1, wherein the distal end of the first ridge segment is terminated proximally of the distal end of the rigid lumen portion.

7. The cartridge of claim 1, wherein the proximal end of the first ridge segment and the distal end of the second ridge segment are in contact with one another when the first wing and the second wing are in the closed position.
